(19) United States
(12) Patent Application Publication
(10) Pub. No.: US 2006/0067943 A1
(43) Pub. Date: Mar. 30, 2006

(54) Stabilization of alum-adjuvanted immunologically active agents

(76) Inventors: Yuh-Fun Maa, Milbrae, CA (US); Scott Sellers, San Mateo, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/237,200

(22) Filed: Sep. 27, 2005

Related U.S. Application Data
(60) Provisional application No. 60/614,161, filed on Sep. 28, 2004.
(60) Provisional application No. 60/649,275, filed on Jan. 31, 2005.

(51) Int. Cl. A61K 48/00; A61K 39/00; A61K 39/12; A61K 39/02; A61K 31/715; A61K 31/7012

(52) U.S. Cl. 424/184.1; 514/53; 514/61; 424/204.1; 424/234.1; 514/44

(57) ABSTRACT

A composition and method for formulating and delivering an adjuvanted immunological active agent, especially a vaccine, wherein adjuvant coagulation and concomitant loss of vaccine efficacy enhancement is mitigated or avoided. The adjuvanted, immunologically-active agent can be subjected to freezing, drying, freeze-drying, or lyophilization, and when reconstituted, retains a high level of potency. The present invention further provides for a composition and method for formulating and delivering a stable, adjuvanted, immunologically-active agent capable of being deposited on a transdermal delivery device or microprojection or array thereof.

### CROSS-REFERENCE TO RELATED APPLICATIONS

This application claims the benefit of U.S. Provisional Application No. 60/614,161, filed Sep. 28, 2004 and 60/649,275 filed Jan. 31, 2005.

### FIELD OF THE PRESENT INVENTION

The present invention relates generally to immunologically active agent compositions and methods for formulating and delivering such compositions. More particularly, the invention relates to compositions of and methods for formulating and delivering alum-adjuvanted immunologically active agents.

### BACKGROUND OF THE INVENTION

As is well known in the art, immunologically active agents, especially antigenic agents or vaccines, can comprise whole viruses (live, attenuated viruses) and bacteria, polysaccharide conjugates, proteins or nucleic acids. Other antigenic agents are composed of synthetic, recombinant, or purified subunit antigens.

Subunit (or split-virion) vaccines are designed to include only the antigens required for protective immunization, and are considered to be safer than whole-inactivated or live attenuated vaccines. Subunit vaccines alone, may, however, exhibit weaker immunogenicity. One solution to the potentially weaker immunogenicity of such vaccines is to formulate the subunit vaccines with adjuvants, which enhance the specific immune response.

Immunologically active agents are typically administered either orally or by injection, and more recently, by transdermal delivery. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Numerous transdermal agent delivery systems and apparatus have been developed that employ tiny skin piercing elements to enhance transdermal agent delivery. Examples of such systems and apparatus are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin, and thus enhance the agent flux. The piercing elements generally extend perpendicularly from a thin, flat member, such as a pad or sheet. The piercing elements are typically extremely small, some having a microprojection length of only about 25-400 microns and a microprojection thickness of only about 5-50 microns.

Some transdermal agent delivery systems may include a drug reservoir that contains a high concentration of an active agent. The reservoir is adapted to contact the skin, which enables the agent to diffuse through the skin and into the body tissues or bloodstream of a patient.

Recent improvements in transdermal agent delivery systems include systems, methods and formulations wherein the active agent to be delivered is coated on the microprojections instead of contained in a physical reservoir. This eliminates the necessity of a separate physical reservoir and developing an agent formulation or composition specifically for the reservoir. U.S. Patent Application Publications Nos. 2004/0062813 (Cormier et al), and 2004/0096455 (Maa et. al.), the disclosures of which are fully incorporated by reference herein, disclose compositions of and methods for formulating and delivering active agents by including the active agent(s) in a coating that is disposed on the microprojections.

As noted in the above-referenced applications, the agent formulation and method of coating the formulation on the microprojections are important factors in transdermal delivery via coated microprojections. Indeed, if a vaccine is employed in the agent formulation that is unstable or does not have sufficient shelf-life, the vaccine may not, and in many instances, will not have the desired (or required) effectiveness.

Accordingly, stabilization of immunologically active agents, e.g. vaccines, is an important step in assuring efficacy of the agents; particularly, when the mode of delivery of the agent is via a transdermal delivery device having a plurality of coated microprojections.

As is known in the art, polypeptide based vaccines, subunit vaccines, and killed viral and bacterial vaccines generally elicit a predominantly humoral response. Replicating vaccines (e.g., live, attenuated viruses, such as polio and smallpox vaccines) result in the most effective humoral and cellular immune responses. A similar broad immune response spectrum can be achieved by DNA vaccines.

To enhance specific immune response to immunologically active agents, adjuvants, such as aluminum salts, are typically added. Adjuvants have diverse mechanisms of action and are typically selected based upon the route of administration and type of immune response (e.g. antibody, cell-mediated, mucosal, etc) that is desired for the particular immunologically active agent.

The stability of alum-adjuvanted immunological active agents, especially vaccines, has, however, been problematic. Alum-adjuvanted immunologically active agents, especially vaccines, tend to lose potency upon freezing and drying. The stability problem of aluminum salt gels upon freeze-thawing is thought to be due to the fact that ice crystals (formed upon freezing) force alum particles to overcome inter-molecular repulsion, thereby producing strong inter-particle attraction. It is thought that this generally applies to any mechanism or system wherein alum becomes concentrated. Thus, in a suspension of alum, wherein alum particles are brought together in close proximity such that inter-molecular repulsive forces are overcome, the alum particles tend to coagulate or agglomerate.

The consequence of freezing or drying, particularly freeze-drying or lyophilizing alum-adjuvanted immunologically active agent formulations, especially vaccines, often results in immunogenicity loss because antigen molecules adsorbed onto the surface of the alum particle cannot be released from within the coagulated formulation matrix.

It would therefore be desirable to provide compositions of and methods for formulating and delivering stable, alum adjuvanted immunologically active agents, and in particular, vaccines, wherein the compositions of and methods for formulating and delivering the agents prevent large-scale alum coagulation, and preserve the potency and immunogenicity of the alum-adjuvanted agent formulations upon drying.

It would be further desirable to provide a stable, alum-adjuvanted immunologically active agent delivery device and method, wherein a stable, alum-adjuvanted immunologically agent-containing formulation is coated onto a transdermal delivery device having a plurality of skin-piercing microprojections that are adapted to deliver the agent through the skin of a subject, and wherein the compositions of and methods for formulating the alum-adjuvanted immunologically active agent preserve the potency and immunogenicity of the alum-adjuvanted immunologically active agent.

A “minimize volume” theory is proposed to reduce alum particle coagulation in Maa, et al., J. Pharm. Sci., 92: 319-332 (2003). This reference describes the mechanism of alum coagulation upon freezing and drying, and its relationship to vaccine potency loss. The reference compares various vaccine dehydration processes and their effects upon vaccine efficacy when reconstituted. The reference does not, however, teach, suggest or disclose an adjuvant formulation, or process therefore, directed to minimizing or mitigating the potency loss of such freezing and drying upon alum-adjuvanted vaccines.

Other references have disclosed methods directed to the freeze-drying process itself in attempts to stabilize adjuvanted vaccines. For example, U.S. Patent Application Pub. No. 2003/0202978 discloses compositions of and methods for formulating and delivering a powdered pharmaceutical formulation. Neither the noted publication, nor any other known reference, however, discloses a formulation of, or technique for, stabilizing alum-adjuvanted vaccines by thin-film coating and drying at ambient temperatures.

It is therefore an object of the present invention to provide compositions of and methods for formulating and delivering a stable, adjuvanted immunologically active agents that can be dried, and readily reconstituted and administered in an immunologically (or biologically) effective amount.

It is another object of the present invention to impart specific physical characteristics in stable, alum-adjuvanted immunologically active agent formulations.

It is another object of the present invention to provide compositions of and methods for formulating and delivering stable, dried alum-adjuvanted vaccine formulations, wherein the formulations do not exhibit significant immunogenicity loss caused by alum-mediated coagulation of the formulation matrix and resultant decrease in available antigen molecules.

It is yet another object of the present invention to provide compositions of and methods for formulating and delivering stable, alum-adjuvanted immunologically active agents, wherein the compositions and methods for formulating such compositions, maximize or optimize efficacy of the immunologically active agents.

It is a further object of the present invention to provide compositions of and methods for formulating and delivering stable adjuvanted immunologically active agents, which compositions are suitable for deposit onto a surface (or a delivery device) as a thin-film, and wherein the compositions and methods for formulating and delivering such compositions maximize or optimize efficacy of the immunologically-active agents.

It is a further object of the present invention to provide compositions of and methods for formulating and delivering stable adjuvanted immunologically active agents, which can be readily employed as a thin-film coating, or as a thin-film, multi-layer coating system on a delivery device, and wherein the thin film coating or coating system maximizes or optimizes efficacy of the immunologically-active agents.

It is yet another object of the present invention to provide compositions of, and methods for formulating and delivering transdermally, stable adjuvanted immunologically active agents, wherein the immunologically active agent can be readily employed as a thin-film coating, or as a thin-film, multi-layer coating system deposited on a plurality of microprojections of a transdermal delivery device, and wherein the thin film coating or coating system maximizes or optimizes efficacy of the immunologically-active agent.

### SUMMARY OF THE INVENTION

In accordance with the above objects and those that will be mentioned and will become apparent below, in one embodiment of the invention, there are provided compositions of and methods for formulating and delivering stable, alum-adjuvanted immunologically active agents, especially vaccines, wherein alum-mediated coagulation and concomitant loss of vaccine efficacy enhancement is mitigated or avoided. The present invention also provides for compositions of and methods for formulating and delivering stable, adjuvanted, immunologically-active agents, especially vaccines, which compositions can be subjected to freezing, drying, freeze-drying, or lyophilization, and when reconstituted, retain a high level of potency.

The present invention further provides for compositions of and methods for formulating and delivering stable, adjuvanted, immunologically-active agents that can be readily deposited onto to a surface (including a delivery device) and dried thereon at ambient temperatures. The present invention further provides for compositions of and methods for formulating and delivering stable, adjuvanted, immunologically-active agents capable of being deposited onto a surface as a thin-film single layer coating or as a thin-film multi-layer coating. Such coatings are particularly suitable for transdermal delivery using a microprojection delivery device, wherein the immunologically active agent is included in a biocompatible coating that is coated on at least one stratum-corneum piercing microprojection, more preferably, a plurality of stratum-corneum piercing microprojections.

The compositions of and methods for formulating and delivering stable, adjuvanted, immunologically-active agents of the present invention allow the production of a thin-film multi-layered coating, having a total thickness in the range of 5-100 microns, more preferably, in the range of 10-50 microns. According to the invention, the alum-containing thin-film layers prevent alum particles from coagulating into a large-scale formulation matrix.

In one embodiment, the method for forming an alum-adjuvanted immunologically active agent formulation that is capable of being disposed on a delivery surface, and/or capable of being dried and reconstituted without significant loss of potency comprises the following steps: (i) preparing a suspension of alum in a suitable solvent, wherein the alum concentration is less than 3%; (ii) adding an amorphous carbohydrate sugar to the alum suspension; (iii) optionally, adding a viscosity-enhancing agent, such as a cellulose or starch, to yield a solution viscosity preferably below 30 centipoise (cP); (iv) adding a film-forming agent, for example, a polyacarboxylic acid; and (v) adding an immunologically active agent to the alum suspension to form an alum-adjuvanted immunologically active agent solution. Preferably, the immunologically active agent is prepared by conventional concentrating and buffering.

In one embodiment, the resultant solution of alum-adjuvanted immunologically active agent is spray-dried, air-dried, spray-freeze dried, freeze-dried or lyophilized to stabilize the solution for storage or distribution. When reconstituted, the potency and immunological response of the immunologically active agent is maximized.

In another embodiment, the resultant solution of alum-adjuvanted immunologically active agent is contained in an agent formulation that is adapted to coat a microprojection delivery device or at least one stratum-corneum piercing microprojection, more preferably, a plurality of stratum-corneum piercing microprojections, or an array thereof. Preferably, the coating process is carried out in a series of coating steps, with a drying step between each coating step. To optimize formation of the desired thin-film of the present invention, the drying time between each drying cycle is preferably long enough to ensure drying of each layer.

In one embodiment, the agent formulation is stabilized on the delivery device or microprojection(s) by drying at ambient temperatures, preferably, in the range of about 15 and 50° C., more preferably, in the range of about 20 and 30° C. However, various temperatures and humidity levels can be employed to dry the coating solution.

Preferably, each coating step results in a layer or coating thickness in the range of about 0.5-5 microns, more preferably, in the range of about 1-3 microns. The aggregate coating thickness is preferably be no more than about 3 microns, more preferably, in the range of about 0.5-5 microns, even more preferably, in the range of about 1-3 microns.

In some embodiments, wherein a vacuum chamber-type drying apparatus is employed, the drying time is preferably in the range of 0.5-360 minutes, more preferably, in the range of 1-100 minutes, under conditions of 0-30% relative humidity and below ambient pressure.

In some embodiments of the invention, the immunologically active agent comprises an antigenic agent or vaccine selected from the group consisting of viruses and bacteria, protein-based vaccines, polysaccharide-based vaccine, and nucleic acid-based vaccines.

Suitable immunologically active agents thus include, without limitation, antigens in the form of proteins, polysaccharide conjugates, oligosaccharides, and lipoproteins. These subunit vaccines include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre-bS1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention. The noted vaccines include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be delivered according to the methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA.

In a preferred embodiment of the invention, the immunologically active agent comprises an influenza vaccine. More preferably, in such embodiments, the immunologically active agent comprises a split-virion influenza vaccine.

In accordance with a further embodiment of the invention, the apparatus for transdermally delivering an immunologically active agent comprises a microprojection member that includes a plurality of microprojections that are adapted to pierce through the stratum corneum into the underlying epidermis layer, or epidermis and dermis layers, the microprojection member having a biocompatible coating disposed thereon that includes a stable, alum-adjuvanted immunologically active agent.

In accordance with one embodiment of the invention, the method for delivering a stable, alum-adjuvanted immunologically active agent comprises the following steps: (i) providing a microprojection member having a plurality of microprojections, (ii) providing a bulk immunologically active agent, (iii), preparing a suspension of less than about 3% alum in solvent; (iv) adding at least one amorphous carbohydrate sugar to the alum suspension, (v) optionally, adding a viscosity-enhancing agent, such as carboxymethyl cellulose, hydroxyethyl cellulose, or a hydroxymethyl starch; (vi) adding a film-forming agent; (vii) forming a biocompatible coating formulation that includes the alum suspension and the immunologically active agent, (viii) coating the microprojection member with the biocompatible coating formulation to form a biocompatible coating; (ix) stabilizing the biocompatible coating by drying, wherein a thin-film coating is established; and (x) applying the coated microprojection member to the skin of a subject.

In accordance with some embodiments of the present invention, the steps (i) through (vii) are then followed by the steps of: (a) stabilizing the resultant biocompatible coating formulation by drying, freeze-drying or lyophilizing to form a stabilized biocompatible coating formulation; (b) reconstituting the biocompatible coating formulation with a solvent (e.g., water) to form a biocompatible coating formulation (including the immunologically active agent); (c) coating a microprojection member with the reconstituted biocompatible coating formulation to form a biocompatible coating; (d) stabilizing the biocompatible coating by drying, wherein a thin-film coating is established; and finally (e) applying the coated microprojection member to the skin of a subject. Preferably the coating is implemented as a thin-film to preserve the immunogenicity and adjuvanticity of the immunologically active agent and alum, respectively.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of a dried alum adsorbed immunologically active agent, prepared by a formulation and method of the prior art, and illustrating regions of alum-mediated coagulation (shown as dark areas in FIG. 1);

FIG. 2 is an illustration of a dry alum-adjuvanted immunologically active agent formulation of the present invention, showing a significant minimization of coagulated alum (again represented by the dark areas), compared to the prior art agent formulation of FIG. 1;

FIG. 3 is an illustration of an alum-adjuvanted immunologically active agent formulation, implemented as a thin film coating system of the present invention;

FIG. 4 is a flow chart illustrating one embodiment of a method for formulating an alum-adjuvanted immunologically active agent of the present invention;

FIG. 5 is a perspective view of a microprojection array upon which a biocompatible coating having an alum-adjuvanted immunologically active agent formulation of the invention can be deposited;

FIG. 6 is a perspective view of the microprojection array of FIG. 5 with a biocompatible coating deposited on the microprojections;

FIG. 6A is a cross sectional view of a single microprojection 10 taken along line 6A-6A in FIG. 6;

FIG. 7 is a view of a skin proximal side of a microprojection array illustrating the division of the microprojection array into various portions;

FIG. 8 is a side sectional view of a microprojection array illustrating an alternative embodiment, wherein different biocompatible coatings are applied to different microprojections; and

FIG. 9 is a side sectional view of a microprojection array having an adhesive backing.

### MODES FOR CARRYING OUT THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an immunologically active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “stable”, as used herein to refer to a formulation, or a coating, means the formulation is not subject to undue chemical or physical decomposition, breakdown, or inactivation. “Stable” as used herein to refer to a coating also means mechanically stable, i.e., not subject to undue displacement, or loss, from the surface upon which the coating is deposited.

The term “co-delivering”, as used herein, means that at least one supplemental agent is administered transdermally either before the agent is delivered, before and during transdermal flux of the agent, during transdermal flux of the agent, during and after transdermal flux of the agent, and/or after transdermal flux of the agent. Additionally, two or more immunologically active agents may be formulated in the biocompatible coatings of the invention, resulting in co-delivery of different immunologically active agents.

The term “immunologically active agent”, as used herein, refers to a composition of matter or mixture containing an antigenic agent and/or a “vaccine” from any and all sources, which is capable of triggering a beneficial immune response when administered in an immunologically effective amount. A specific example of an immunologically active agent is an influenza vaccine.

Further examples of immunologically active agents include, without limitation, viruses and bacteria, protein-based vaccines, polysaccharide-based vaccines, and nucleic acid-based vaccines.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

The term “coating formulation”, as used herein, is meant to mean and include a freely flowing composition or mixture, in a liquid or a solid state, which is employed to coat a delivery surface, including a plurality of microprojections and/or arrays thereof.

The term “biocompatible coating”, as used herein, means and includes a “coating formulation” which has sufficient adhesion characteristics and no or minimal adverse interactions with the immunologically active agent.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration. The microprojection member can also be formed in other known manners, such as by forming one or more strips having microprojections along an edge of each of the strip(s) as disclosed in U.S. Pat. No. 6,050,988; which is hereby incorporated by reference in its entirety.

Microprojection members that can be employed with the present invention include, but are not limited to, the members disclosed in U.S. Pat. Nos. 6,083,196, 6,050,988 and 6,091,975, and U.S. Pat. Pub. No. 2002/0016562, which are incorporated by reference herein in their entirety. As will be appreciated by one having ordinary skill in the art, where a microprojection array is employed, the dose of the immunologically active agent that is delivered can also be varied or manipulated by altering the microprojection array (or patch) size, density, etc.

### DETAILED DESCRIPTION OF THE INVENTION

As is known to the art, the desired physiological response to an immunologically active agent, especially a vaccine, is the stimulation of an immune response. An immune response, especially production of antibodies, is enhanced by formulating the vaccines with supplemental components, such as adjuvants, which enhance specific immune responses to the agents. Modern immunologically active agents particularly vaccines and more particularly, subunit-type, or split virion vaccines, are typically formulated with such adjuvants to improve the efficacy and potency of the agent.

As is known in the art, advantages associated with the use of adjuvants in a vaccine formulation include the ability to: (1) direct and optimize immune responses appropriate for the vaccine; (2) facilitate mucosal delivery of vaccines; (3) promote cell-mediated immune responses; (4) enhance the immunogenicity of weaker antigens; (5) reduce the amount of antigen and/or the frequency of administration required to provide protective immunity; and (6) improve the efficacy of vaccines in individuals with weak immune responses.

Adjuvant mechanisms of action typically include: (1) increasing the biological or immunological storage life of vaccine antigens; (2) improving antigen delivery to antigen presenting cells; (3) improving antigen processing by antigen presenting cells; and (4) inducing the production of immunomodulatory cytokines.

Mineral adjuvants are one widely-used class of adjuvants. Such adjuvants include, for example, salts of metals such as cerium, zinc, iron, aluminum and calcium. Aluminum salts, phosphate and hydroxide in particular, are widely employed. Indeed, such salts are employed in more than 50% of the commercial vaccine products including Hepatitis B vaccine (Alum-HBsAg) and diphtheria and tetanus toxoid vaccine (Alum-DT). As used herein, unless otherwise clear from the context, the term “alum” is used to encompass both aluminum hydroxide and aluminum phosphate.

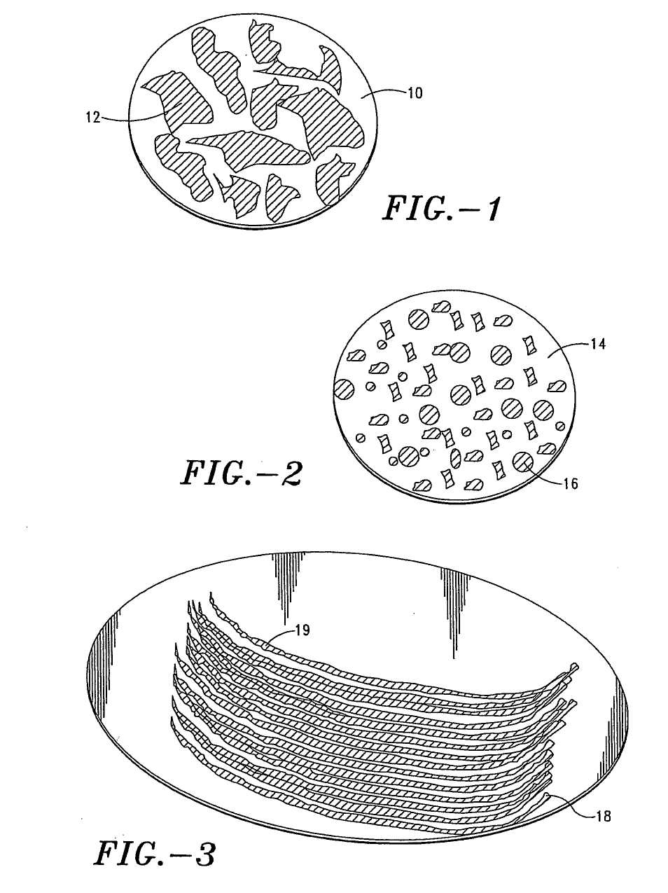

Referring to FIG. 1, there is illustrated an alum matrix 10 prepared by a formulation and method of the prior art. The formulation for which FIG. 1 is representative comprises: Al(OH)₃ (2.9%)+trehalose (4.1%), AlPO3 (3.0%)+dextran (1.2%), or AlPO3 (5.0%)+mannitol (2.2%) and was prepared by air-drying or freeze-drying (see, Maa et. al., Pharm. Res., Vol. 20, No. 7, July 2003, pp. 969-977).

The dark regions 12 shown in FIG. 1 represent a large scale alum-mediated coagulation. Such large scale coagulation results in a high percentage of antigen being adsorbed at the alum surface and trapped in the coagulate and, hence, is unavailable to elicit the desired immunological response.

As indicated above, the present invention comprises compositions of and methods for formulating and delivering stable alum-adjuvanted immunological active agents, especially vaccines, wherein adjuvant-mediated coagulation and concomitant loss of antigenic agent efficacy enhancement is mitigated or avoided. The present invention also provides for compositions and methods for formulating and delivering stable, alum-adjuvanted, immunologically active agents, especially vaccines, which can be subjected to freezing, drying, freeze-drying, or lyophilization, and when reconstituted, retain a high level of potency. In a preferred embodiment of the present invention, the immunologically active agent comprises a vaccine, and the adjuvant includes an aluminum hydroxide, an aluminum phosphate, and mixtures thereof.

Referring now to FIG. 2, there is shown a matrix 14 reflective of an alum-adjuvanted immunologically active agent formulation of the present invention, following drying and reconstitution. The formulation for which FIG. 2 is representative is further described in Example 1 and was prepared by spray drying and spray freeze drying.

It can be seen that, compared to the matrix shown in FIG. 1, the dark regions 16, which are representative of alum-mediated coagulation, occupy significantly less area. As a result, a greater percentage of antigen is available to elicit the desired immunological response.

In one embodiment, the present invention thus comprises compositions of and methods for formulating and delivering stable, adjuvanted, immunologically-active agents which can be readily deposited onto to a surface (or a delivery device) and dried thereon at ambient temperatures. The present invention further provides for compositions of and methods for formulating and delivering stable, adjuvanted, immunologically-active agents capable of being deposited on a surface (or delivery device) as a thin-film coating.

The thin-film coating can comprise a single layer or be multi-layered. The single and multi-layer thin-film coatings are particularly suitable for transdermal delivery using a microprojection based delivery device. With such a device, the agent, e.g., a vaccine, is included in a biocompatible coating that is coated on at least one, more preferably, a plurality of stratum-corneum piercing microprojections.

In one embodiment of the present invention, the compositions of and methods for formulating and delivering stable, adjuvanted, immunologically-active agents allow the production of a thin-film multi-layered coating, having a total thickness in the range of 5-100 microns, more preferably, in the range of 10-50 microns. According to the invention, the alum-containing thin-film layers prevent alum particles from coagulating into a large-scale formulation matrix. Upon dissolution within the skin by the interstitial fluid, the vaccine antigen can be readily released and, hence, administered. The antigenic agent's immunogenicity and the alum's adjuvanticity are also preserved in the coating formulation, whereby the resultant efficacy of the antigenic agent is improved.

Referring to FIG. 4, in one embodiment, the method for forming an alum-adjuvanted immunologically active agent that is capable of being disposed on a delivery surface and/or capable of being dried and reconstituted without significant loss of potency comprises the following steps: (i) preparing a suspension of alum in a suitable solvent, wherein the alum concentration is less than 3% (20); (ii) adding an amorphous carbohydrate sugar (i.e., a bulking agent and protein stabilizer) to the alum suspension (21); (iii) optionally, adding a viscosity-enhancing agent, such as a cellulose or starch, to yield a solution viscosity (22); (iv) adding a film-forming agent, for example, a polyacarboxylic acid (23); and (v) adding a bulk immunologically active agent to the alum suspension (24). According to the invention, the immunologically active agent can be prepared by concentrating and buffering, as known in the art.

In one embodiment, the resultant solution of alum-adjuvanted immunologically active agent can then be spray-dried, air-dried, spray-freeze dried, freeze-dried or lyophilized to stabilize it for storage or distribution (26). When reconstituted, the potency and immunological response of the immunologically active agent is maximized.

In another embodiment, the resultant solution of alum-adjuvanted immunologically active agent is included in a biocompatible coating formulation (27), which can be employed as a coating on a delivery device, a stratum-corneum piercing microprojection or a plurality of stratum-corneum piercing microprojections or an array thereof. Preferably, the coating process is carried out in a series of coating steps, with a drying step between each coating step. To optimize formation of the desired thin-film of the present invention, the drying time between each drying cycle should be long enough to ensure drying of each layer to avoid mixing the new coating with previously un-dried coating(s).

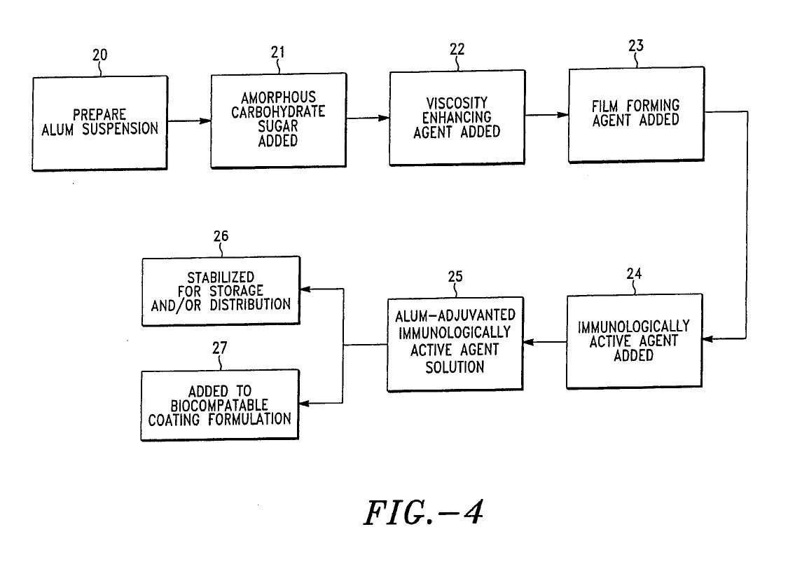

Beneficial results achieved by the desired thin-film coatings of alum-adjuvanted formulations and methods of the present invention are illustrated diagrammatically in FIG. 3. As illustrated in FIG. 3, coagulation of the formulation matrix 18 is minimized by the thin-film coating process of the present invention, which provides a thin film array (i.e., layers) 19 of the alum-adjuvanted immunologically active agent formulation. The formulation for which the matrix shown in FIG. 3 is representative is further described in Example 2, and was prepared by film coating.

The method and materials used to formulate the alum-adjuvanted immunologically active agent of the present invention are described in more detail below.

Alum Suspension Preparation (20)

A suspension of aluminum hydroxide or aluminum phosphate is prepared by adding about 0.0001% to 3%, preferably about 0.05 to 2 weight percent aluminum hydroxide or aluminum phosphate to a suitable solvent. Suitable solvents include water and ethanol. The resultant solution viscosity should be in the range of about 1 to 50 cP, more preferably, in the range of about 10 to 30 cP. While aluminum hydroxide and aluminum phosphate are preferred metal compounds, other metal salts, such as calcium phosphate, magnesium hydroxide and aluminum hydroxycarbonate can also be useful.

In addition to alum, other immune response augmenting adjuvants can be formulated with the vaccine antigen to comprise the vaccine. Such adjuvants include, without limitation, algal glucan, β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂O (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; salvo peptide: VQGEESNDK•HCl (IL-1β 163-171 peptide); and threonyl-MDP (Termurtide™): N-acetyl muramyl-L-threonyl-D-isoglutamine, and interleukine 18, IL-2 IL-12, IL-15, Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

Addition of Amorphous Sugar (21)

As is known in the art, amorphous sugar functions as a bulking agent and protein stabilizer. According to the invention, the amorphous sugar can be selected from the general class of glass-forming sugars. In particular, sucrose, melezitose, raffinose, trehalose, stachyose, lactose, maltose and combinations thereof are useful. The sugar is preferably added in a bulking-effective or protein-stabilizing effective amount. On a weight percentage basis, the sugar can be present in a range of about 2 to 40 wt. % percent, more preferably, in a range of about 10 to 30 wt. %.

Addition of Viscosity-Enhancing Agent (22)

Optionally, a viscosity-enhancing agent, such as a polymeric material, can be added to the formulation. Examples of suitable polymers include, without limitation, cellulose derivatives, such as hydroxyethylcellulose (HEC), hydroxypropyl-methylcellulose (HPMC), hydroxypropylcellulose (HPC), methylcellulose (MC), hydroxyethylmethylcellulose (HEMC), or ethylhydroxyethylcellulose (EHEC).

The viscosity-enhancing agent, if employed, is added in an amount sufficient to provide a final solution viscosity of preferably less than about 100 cP, more preferably, in the range of about 20-60 cP. According to the invention, the viscosity-enhancing agent can be present in a range of about 0.1-10 wt. %.

Addition of Film-Forming Agent (23)

Many examples of film forming agents exist. Generally, the film-forming agents comprise polycarboxylic acids, and salts thereof, which have a molecule weight in a range of about 1,000-1,000,000 Daltons, more preferably, in the range of about 30,000-500,000 Daltons. Non-limiting examples include polymers of acrylic acid, methacrylic acid, acrylamide, acrylnitrile and others. Copolymers of carboxylic acids are also suitable. Preferred polymers are polyacrylates, such as those commercially available under the trademarks CARBOPOL® and ACUSOL®, and hydroypropylmethy cellulose (HPMC), hydroxylethyl cellulose (HEC) and polyvinyl alcohol (PVOH).

The film-forming agent is preferably added in a film-forming effective amount. According to the invention, the film-forming agent can be present in a range of about 0.001-10 wt. %, more preferably, in the range of about 0.1 to 5 wt. %.

Addition of Immunologically Active Agents (24)

Immunologically active agents, such as vaccines, are typically prepared in aqueous form, using a suitable carrier, along with suitable adjuvants, excipients, protectants, solvents, salts, surfactants, buffering agents and other components. The immunologically active agent is typically prepared by concentrating and buffering as known in the art.

Suitable immunologically active agents thus include, without limitation, antigens in the form of proteins, polysaccharide conjugates, oligosaccharides, and lipoproteins. These subunit vaccines include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention. The noted vaccines include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be employed according to the formulations and methods of the invention, include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. The nucleic acid can also be coupled with a proteinaceous agent or can include one or more chemical modifications, such as, for example, phosphorothioate moieties.

The alum-adjuvanted immunologically active agent formulations of the present invention can also contain suitable excipients, protectants, solvents, salts, surfactants buffering agents and other components. Examples of such formulations can be found in U.S. Patent Application Nos. 60/600,560 and 10/970,890; the disclosures of which are incorporated by reference herein.

According to the invention, the resultant solution of alum-adjuvanted immunologically active agent (25) can then be spray-dried, air-dried, spray-freeze dried, freeze-dried or lyophilized to stabilize it for storage or distribution (26). When reconstituted, the potency and immunological response of the immunologically active agent is maximized.

In another embodiment, the resultant solution of alum-adjuvanted immunologically active agent (25) is included in a biocompatible coating formulation (27) that can be employed as a coating on a delivery device or a stratum-corneum piercing microprojection or array thereof. Compositions and methods of formulating biocompatible coatings are described in detail in U.S. application Ser. Nos. 10/127,108 and 10/608,304; the disclosures of which are incorporated herein by reference.

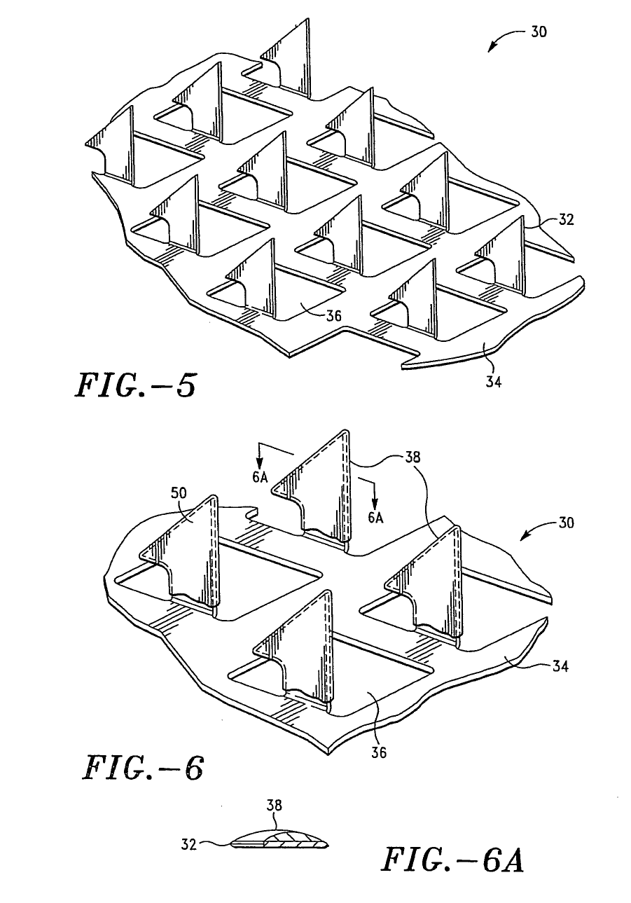

Referring now to FIG. 5, there is shown one embodiment of a stratum corneum-piercing microprojection array for use with the compositions and methods of the present invention. As illustrated in FIG. 5, the microprojection array 30 includes a plurality of microprojections 32. The microprojections 32 extend at substantially a 90° angle from a sheet 34 having openings 36.

As illustrated in FIG. 9, the sheet 34 can be incorporated in a delivery patch having a backing 35 for the sheet 34. The backing 35 can further include an adhesive 36 for adhering the backing 35 and microprojection array 30 to a patient's skin. In this embodiment, the microprojections 32 are formed by either etching or punching a plurality of microprojections 32 out of a plane of the sheet 34.

The microprojection array 30 can be manufactured of metals such as stainless steel, titanium, nickel titanium alloys, or similar bio-compatible materials such as plastics. The microprojection array 30 is preferably constructed of titanium. Metal microprojection members are disclosed in Trautman et al., U.S. Pat. No. 6,038,196; Zuck U.S. Pat. No. 6,050,988; and Daddona et al., U.S. Pat. No. 6,091,975, the disclosures of which are herein incorporated by reference.

Other microprojection members that can be used with the present invention are formed by etching silicon, by utilizing chip etching techniques or by molding plastic using etched micro-molds. Silicon and plastic microprojection members are disclosed in Godshall et al., U.S. Pat. No. 5,879,326, the disclosure of which is incorporated herein by reference.

With such microprojection devices, it is important that the biocompatible coating having the immunologically active agent is applied to the microprojections homogeneously and evenly, preferably limited to the microprojections themselves. This enables dissolution of the agent in the interstitial fluid once the device has been applied to the skin and the stratum corneum pierced. Additionally, a homogeneous coating provides for greater mechanical stability both during storage and during insertion into the skin. Weak and/or discontinuous coatings are more likely to flake off during manufacture and storage, and to be wiped of the skin during application.

Additionally, optimal stability and shelf life of the agent is attained by a biocompatible coating that is solid and substantially dry. However, the kinetics of the coating dissolution and agent release can vary appreciably depending upon a number of factors. It will be readily appreciated that in addition to being storage stable, the biocompatible coating should permit desired release of the agent.

Referring now to FIG. 6, there is shown the microprojection array 30, wherein the microprojections 32 have been coated with a biocompatible coating 50. According to the invention, the biocompatible coating 50 can partially or completely cover the microprojections 32. The biocompatible coating 50 can be applied to the microprojections before or after the microprojections 32 are formed.

The coating 50 on the microprojections can be formed by a variety of known methods. One such method is dip-coating. Dip-coating can be described as a means to coat the microprojections by partially or totally immersing the microprojections into the immunologically active agent-containing biocompatible coating solution. Alternatively, the entire device can be immersed into the biocompatible coating solution. In many instances, the immunologically active agent within the coating may be very expensive. Thus, it may be preferable to only coat the tips of the microprojections. Microprojection tip coating apparatus and methods are disclosed in Trautman et al., U.S. Patent Application Pub. No. 2002/0132054; the disclosure of which is incorporated herein by reference.

As described in the above-referenced application, the coating apparatus only applies a coating to the microprojections and not to the substrate/sheet from which the microprojections project. This may be desirable where the cost of the immunologically active agent is relatively high and therefore the biocompatible coating containing the immunologically active agent should only be disposed onto parts of the microprojection array that will pierce the subject's stratum corneum layer. This coating technique has the added advantage of naturally forming a smooth coating that is not easily dislodged from the microprojections during skin piercing. The smooth cross section of the microprojection tip coating is more clearly shown in FIG. 6A.

Other coating techniques, such as microfluidic spray or printing techniques can also be used to precisely deposit a coating 38 on the tips of the microprojections 32, as shown in FIG. 6.

Additional coating methods, which may be employed in the practice of the present invention, include spraying the coating solution on the microprojections 32. Spraying can encompass formation of an aerosol suspension of the coating composition. In one embodiment, an aerosol suspension forming a droplet size of about 10 to about 200 picoliters is sprayed onto the microprojections and then dried.

According to the invention, the microprojections 32 can further include means adapted to receive and/or increase the volume of the coating 38, such as apertures (not shown), grooves (not shown), surface irregularities (not shown), or similar modifications, wherein the means provides increased surface area upon which a greater amount of coating may be deposited.

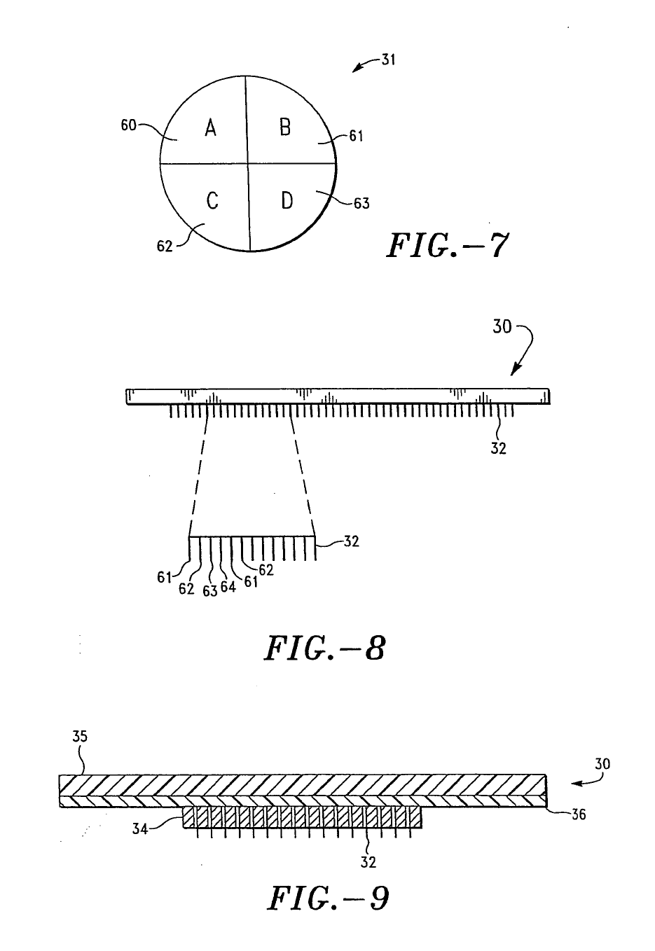

Referring now to FIG. 7, there is shown an alternative embodiment of a microprojection array 31. As illustrated in FIG. 7, the microprojection array 31 can be divided into portions, illustrated at 60-63, wherein a different coating is applied to each portion, thereby allowing a single microprojection array to be utilized to deliver more than one beneficial agent during use.

Referring now to FIG. 8, there is shown a cross-sectional view of a microprojection array 30 having a plurality of microprojections 32, wherein the microprojections 32 are coated in a series with a different biocompatible coating and/or a different immunologically active agent, as indicated by reference numerals 61-64. That is, separate coatings are applied to a desired number of individual microprojections 32.

In a preferred embodiment, pattern coating is employed to coat the microprojections 32. The pattern coating can be applied using a dispensing system for positioning the deposited liquid onto the surface of the microprojection array. The quantity of the deposited liquid is preferably in the range of 0.1 to 20 nanoliters per microprojection. Examples of suitable precision-metered liquid dispensers are disclosed in Tisone, U.S. Pat. Nos. 5,916,524, 5,743,960, 5,741,554 and 5,738,728; the disclosures of which are incorporated herein by reference.

Microprojection coating solutions can also be applied using ink jet technology using known solenoid valve dispensers, optional fluid motive means and positioning means which are generally controlled by use of an electric field. Other liquid dispensing technology from the printing industry or similar liquid dispensing technology known in the art can be used for applying the pattern coating of this invention.

In a preferred embodiment, the process of applying a biocompatible coating containing an alum-adjuvanted immunologically active agent to at least one stratum-corneum piercing microprojection of a microprojection member, more preferably, to a plurality of such stratum-corneum piercing microprojections, includes the step of further stabilizing the biocompatible coating by the drying at ambient temperatures.

Preferably, the coating process is carried out in a series of coating steps, with a drying step between each coating step. To optimize formation of the desired thin-film of the present invention, the drying time between each drying cycle should be long enough to ensure drying of each layer.

Each coating step preferably results in a layer or coating thickness in the range of about 0.5-5 microns, more preferably, in the range of about 1-3 microns. The total or aggregate coating thickness should be no more than about 3 microns, preferably, in the range of about 5-100 microns, more preferably, in the range of about 10-50 microns. In some embodiments, wherein a vacuum chamber-type drying apparatus is employed, the drying time is preferably in the range of 0.5-360 minutes, more preferably, in the range of 1-100 minutes, under conditions of 0-3% relative humidity and below ambient pressure.

In one embodiment of the present invention, the biocompatible coating containing the alum-adjuvanted immunologically active agent formulation is dried at ambient room temperatures to achieve the desired thin-film coating and concomitant absence of antigen-masking coagulation. One way to achieve this is to dry the solution using a spray-drying type drying apparatus. In such an apparatus, a fine mist of solubilized material (e.g., immunologically active agent solution) is introduced into a large conical chamber where it comes into contact with air that has been heated to a range of about 60-250° C. preferably about 80-150° C. Exact conditions of temperature, pressure, humidity and residence time depend on the material or agent being dried. For the immunologically active agents of the present invention, the drying condition is preferably conducted at an inlet temperature in the range from about 60° C. to about 150° C., more preferably, in the range from about 80° C. to about 120° C. Suitable feed rates are in the range from about 1 mL/min to about 20 mL/min, more preferably, about 5-10 mL/min. Humidity may range from about 10-50 percent.

One suitable drying apparatus is disclosed in U.S. Patent Application No. 60/572,861, filed May 19, 2004 [Docket Number ALZ5134]; the disclosure of which is incorporated by reference herein.

As will be appreciated by one have ordinary skill in the art, the noted formulations and processes of the invention can be modified and adapted to formulate various vaccine source materials and forms thereof. For example, the process could be adapted to formulate hepatitis-B, diphtheria and tetanus toxoids.

According to the invention, a multitude of immunologically active agents or vaccines can be subjected to the formulation process and methods of the invention to provide highly stable vaccine formulations. In a preferred embodiment of the invention, the immunologically active agent comprises an influenza vaccine, more preferably, a split-varion influenza vaccine.

### EXAMPLES

The following studies and examples illustrate the formulations, methods and processes of the invention. The examples are for illustrative purposes only and are not meant to limit the scope of the invention in any way.

### Example 1

An aluminum hydroxide adsorbed hepatitis B surface antigen (HBsAg) solution was formulated with the composition as summarized in Table 1.

TABLE I

| Ingredient | Weight Percentage |
|---|---|
| AL(OH)₃/HBsAg | 3 |
| Amorphous sugar (trehalose) | 10 |
| Viscosity-enhancing agent (dextran of | 3 |
| 37,000 dalton) | |

This liquid formulation was spray dried or spray freeze dried.

Spray Drying

A bench-top spray dryer (Büchi) was used with the following conditions: drying air inlet temperature of 130-140° C., liquid feed of 3.5 mL/min, and drying air outlet temperature of 80-85° C. The resulting powder has a particle size distribution of D_{10%}=1.2 μm, D_{50%}=3.5 μm, and D_{90%}=5.8 μm.

Spray Freeze Drying

The liquid formulation was sprayed using an ultrasonic atomizer (60 kHz, Sono-Tek Corporation, Milton, N.Y.) at a feed rate of 1.5 mL/min into liquid nitrogen contained in a stainless steel pan. The pan was transferred to a lyophilizer (DuraStop, FTS Systems, Stone Ridge, N.Y.) with pre-chilled shelves at −55° C.

Primary drying was performed at −10° C. for 10 hours and secondary drying at 15° C. and then 25° C. for 5 hours each. Throughout the cycle, the temperature ramping rate was set at 1° C./minute and the chamber vacuum at 100 mTor. The resulting powder has a particle size distribution of D_{10%}=25 μm, D_{50%}=40 μm, and D_{90%}=60 μm.

Alum Coagulation Analysis

Alum coagulation was evaluated under optical microscopy on the reconstituted powders prepared by spray drying and spray freeze drying. In contrast to the unprocessed AL(OH)₃/HBsAg liquid displaying a sandy texture under optical microscopic image, both the reconstituted powder formulations also show the same sandy morphology with no obvious particles, suggesting minimal alum coagulation in the powder formulation.

Potency and Antigenicity Analysis

The potency/antigenicity of HBsAg in the powder formulation was determined by a quantitative enzyme immune assay using AUSZYME® Monoclonal kit (Abbot Laboratory, Abbott Park, Ill.). In this study, HBsAg potency of the reconstituted powder formulations were comparable to that the unprocessed HBsAg starting material.

### Example 2

An aluminum hydroxide adsorbed hepatitis B surface antigen (HBsAg) solution was formulated with the composition as summarized in Table 2.

TABLE 2

| Ingredient | Weight Percentage |
|---|---|
| AL(OH)₃/HBsAg | 3 |
| Amorphous sugar (sucrose) | 5 |
| Viscosity-enhancing agent | 5 |
| (PVP of 50,000 dalton) | |
| Surface active agent (polysorbate 20) | 0.1 |

The resulting solution was characterized and it displayed a viscosity of 40 cps and a contact angle of 40° C. on titanium metal. Dip coating was applied to the tip of an array of 225-μm microprojections (725 microprojections per cm²) by submerging the top 100 μm of the microprojection into this solution. After each dip, the liquid uptake was dried for 10 seconds under the ambient air condition (22° C. and 50% relative humidity). The coating process was repeated 10 times until the thickness of the dry coat was approximately 20 μm.

Alum Coagulation Analysis

The dry coat formulation was reconstituted in water. Optical microscopy was used to measure alum coagulation in the reconstituted solution. Like the unprocessed AL(OH)₃/HBsAg liquid, the reconstituted formulation shows the similar sandy morphology with no obvious particles, suggesting minimal alum coagulation in the dry coat.

Potency and Antigenicity Analysis

The potency/antigenicity of HBsAg in the dry coat formulation was determined by a quantitative enzyme immune assay using AUSZYME® Monoclonal kit (Abbot Laboratory, Abbott Park, Ill.). In this study, HBsAg potency of the reconstituted dry coat formulation were comparable to that the unprocessed HBsAg starting material.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An immunologically active agent-containing formulation, the formulation comprising:
a biologically effective amount of an immunologically active agent;
a stabilizing amount of a carbohydrate sugar; and
an amount of an aluminum salt to minimize the loss of immunogenicity of the immunologically active agent when being subjected to drying.

2. The formulation of claim 1, wherein coagulation of the aluminum salt is minimized to preserve the potency and immunogenicity of the formulation.

3. The formulation of claim 1, wherein the aluminum salt is aluminum hydroxide, aluminum phosphate, or mixtures thereof.

4. The formulation of claim 1, wherein the formulation contains about 0.0001% to 3% weight percent of the aluminum salt based on the total weight of the formulation.

5. The formulation of claim 1, wherein the sugar is selected from the group consisting of sucrose, metezitose, raffinose, trehalose, stachyose, lactose, maltose and combinations thereof.

6. The formulation of claim 1, wherein the formulation contains in the range of about 2 to 40 weight percent of the sugar based on the total weight of the formulation.

7. The formulation of claim 1, further comprising a viscosity-enhancing agent.

8. The formulation of claim 7, wherein the viscosity-enhancing agent is present such that the formulation has a viscosity in the range of about 1 to 50 centipoises.

9. The formulation of claim 1, further comprising a film-forming agent.

10. The formulation of claim 9, wherein the film-forming agent is present in the formulation in the range of about 0.001 to 10 weight percent based on the total weight of the formulation.

11. The formulation of claim 1, wherein the formulation further comprises at least one additional adjuvant.

12. The formulation of claim 1, wherein the immunologically active agent is selected from the group consisting of: virus, bacteria, protein-based vaccine, polysaccharide-based vaccine, nucleic acid-based vaccine and combinations thereof.

13. A device for transdermally delivering an immunologically active agent, the device comprising:
a member having a plurality of stratum corneum-piercing microprojections adapted to transdermally deliver a immunologically active agent; and
a formulation of claim 1 coated on at least one of the microprojections.

14. The device of claim 13, wherein the member has a plurality of stratum corneum-piercing microprojections.

15. The device of claim 13, wherein said member is manufactured from a metal consisting of the group of stainless steel, titanium, nickel titanium alloys, or similar biocompatible materials.

16. A method of preparing an alum-adjuvanted immunologically active agent coating, the method comprising the steps of:
preparing a coating composition comprising one or more aluminum salts in a suitable solvent, wherein the total aluminum salt concentration is less than about 3 weight percent based on the total weight of the coating composition, at least one carbohydrate sugar, an optional viscosity-enhancing agent, and an immunologically active agent;
applying said coating composition to a substrate; and
drying, or allowing to dry, said applied coating composition to prepare said dried coating.

17. The method of claim 16, wherein the method comprises the further step of adding a film-forming agent to the coating composition.

18. The method of claim 16, the method further comprising the step of subjecting the coating composition to a drying process selected from the group consisting of: spray-drying, air-drying, spray-freeze drying, freeze-drying, lyophillization or combinations thereof.

19. The method of claim 18, the method further comprising the step of reconstituting the coating composition with a solvent.

20. The method of claim 19, the method further comprising the step of applying the reconstituted coating composition to at least one stratum-corneum piercing microprojection to form a biocompatible coating.

21. The method of claim 20, wherein the biocompatible coating is applied to the at least one stratum-corneum piercing microprojections by a method selected from the group consisting of dip-coating, microfluidic spray, printing and spraying.

22. The method of claim 21, wherein the biocompatible coating is dried or allowed to dry.

23. The method of claim 21, wherein the coating composition is applied to the at least one stratum-corneum piercing microprojections in a series of applications.

24. The method of claim 23, wherein a drying step is performed between substantially all of the applications.

25. The method of claim 22, wherein the drying step is performed at an ambient temperature.

26. The method of claim 25, wherein the drying time is in the range of about 0.5 to 360 minutes.

27. The method of claim 26, wherein the drying step is performed under conditions of about 0 to 30 percent relative humidity and below ambient pressure.

28. The method of claim 20, wherein the biocompatible coating has a thickness in the range of about 0.5 to 5 microns.

29. The method of claim 23, wherein the aggregate coating thickness is in the range of about 0.5 to 5 microns.

30. A method of transdermally delivering an immunologically active agent, the method comprising:
preparing the alum-adjuvanted immunologically active agent coating of claim 20; and
applying the member so that the immunologically active agent is delivered through the skin of a subject.
